(19) United States
(12) Patent Application Publication
(10) Pub. No.: US 2008/0306047 A1
(43) Pub. Date: Dec. 11, 2008

(54) Use of Aza-Phenylalanine Compounds For Treating Cardiac Arrhythmia

(76) Inventors: Manica Cerne, Ig (SI); Uros Urleb, Ljubljana (SI); Metka V. Budihna, Ljubljana (SI); Gorazd Drevensek, Ljubljana (SI); Luka Peternel, Ljubljana (SI)

Correspondence Address: MANELLI DENISON & SELTER, 2000 M STREET NW SUITE 700, WASHINGTON, DC, 20036-3307, US

(21) Appl. No.: 12/093,974

(22) PCT Filed: Nov. 16, 2006
(86) PCT No.: PCT/EP2006/010994

§ 371 Date: Jul. 24, 2008

(30) Foreign Application Priority Data
Nov. 18, 2005 (GB) 0523539.5

(51) Int. Cl. A61K 31/55; A61P 9/00

(52) U.S. Cl. 514/217.05; 514/217.11; 514/217.03; 514/217.08

(57) ABSTRACT

The present invention relates to the use of certain aza-phenylalanine compounds of formula (I) wherein R₁ denotes amino, alkylamino, dialkylamino, amidino, alkylamidino, N-hydroxyamidino, or N-alkoxyamidino, Y denotes sulfonyl or carbonyl and R2 and R4 independently represent various organic residues, in the treatment or prevention of cardiac arrhythmia and/or in treatment or prevention of diseases in association with ischemic heart conditions.

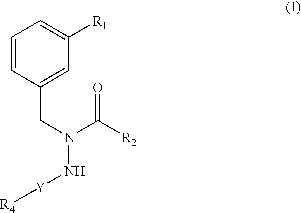

The present invention relates to the use of certain aza-phenylalanine compounds in the treatment or prevention of cardiac arrhythmia.

In one aspect the present invention relates to the use of a compound of formula

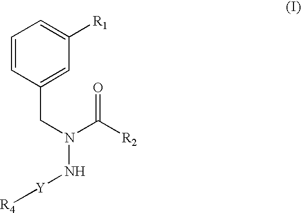

or pharmaceutically acceptable salts or solvates thereof in the preparation of a medicament for the treatment or prevention, e.g. the treatment, of cardiac arrhythmias, whereby in formula I R₁ denotes amino, alkylamino, dialkylamino, amidino, alkylamidino, N-hydroxyamidino, or N-alkoxyamidino,
R₂ denotes a group of formula

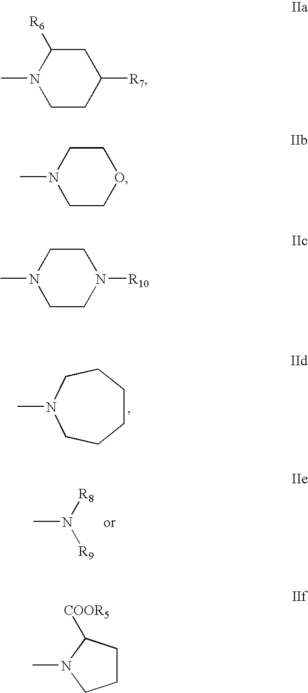

wherein R₅ denotes hydrogen, alkyl or aryl,
R₆ and R₇ each independently denote hydrogen, alkyl or —COOR₁₁,
R₈ and R₉ each independently denote alkyl or cycloalkyl,
R₁₀ denotes hydrogen, alkoxycarbonyl or alkylsulfonyl, and
R₁₁ denotes hydrogen or alkyl,
Y denotes carbonyl or sulfonyl, e.g. represented by a group of

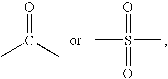

R₄ denotes alkyl, alkoxy or a group of formula

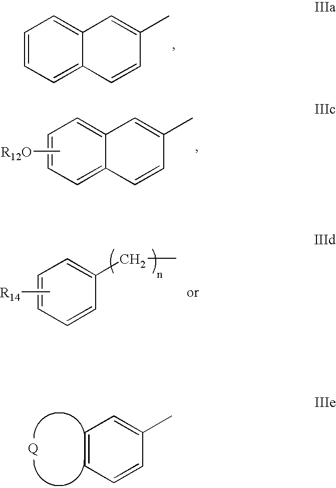

wherein R₁₂ denotes alkyl, R₁₄ denotes alkyl or halogen, n represents an integer number from 0 to 3, preferably from 0 to 1, such as 0, and Q forms together with the phenyl ring to which it is attached a 5- to 7-membered, particularly 6-membered, alicyclic ring having at one position a nitrogen atom substituted by R₁₃, i.e. a group of

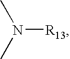

wherein R₁₃ denotes hydrogen, alkyl or acyl

If not otherwise defined herein, any carbon atom containing group may have from 1 to 20 carbon atoms. Alkyl includes branched or unbranched (C₁₋₈)alkyl, such as (C₁₋₄)alkyl, in particular (C₁₋₃)alkyl, e.g. methyl or ethyl. Cycloalkyl includes (C₃₋₈)cycloalkyl, in particular (C₃₋₆)-cycloalkyl, such as cyclohexyl. Alkyl and Cycloalkyl may be unsubstituted or substituted, e.g. one or several fold substituted by for instance halogen such as fluoro. Alkoxy includes alkoxy wherein the alkyl part is as defined above for alkyl. Halogen includes fluoro, chloro and bromo, in particular fluoro and chloro. Aryl includes (C₆₋₁₈)aryl, in particular (C₆₋₁₂)aryl such as phenyl. Alkylamino and dialkylamino includes alkylamino and dialkylamino wherein the alkyl parts are as defined above for alkyl. Alkoxycarbonyl and alkylsulfonyl include alkoxycarbonyl and alkylsulfonyl wherein the alkyl parts are as defined above for alkyl. Acyl includes (C₁₋₁₂)acyl, e.g. (C₁₋₆)acyl such as acetyl. Alkylamidino and N-alkoxy-amidino include alkylamidino and N-alkoxyamidino wherein the alkyl parts are as defined above for alkyl.

In a compound of formula I as defined above, R₁ denotes preferably amidino, alkylamidino, N-hydroxyamidino, or N-alkoxyamidino, e.g. amidino, N-hydroxyamidino or N-alkoxyamidino, and the other variables Y, R₂, R₄, R₅, R₆, R₇, R₈, R₉, R₁₀, R₁₁, R₁₂, R₁₃, R₁₄, Q and n are as defined above.

In a compound of formula I as defined above, R₂ denotes preferably a group of formula IIa or IId and the other variables Y, R₂, R₄, R₅, R₆, R₇, R₈, R₉, R₁₀, R₁₁, R₁₂, R₁₃, R₁₄, Q and n are as defined above. Preferably, R₆ and R₇ independently denote each hydrogen, unsubstituted alkyl or alkyl substituted by halogen such as fluoro. More preferably, R₆ and R₇ independently denote each hydrogen, methyl or trifluoromethyl. Thus, a particularly preferred meaning of R₂ is a group of formula

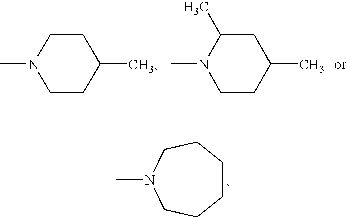

such as a group of formula IId.

In a compound of formula I as defined above, R₄ denotes preferably a group of formula

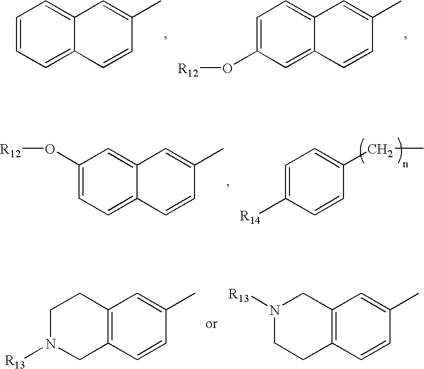

and the other variables Y, R₂, R₄, R₅, R₆, R₇, R₈, R₉, R₁₀, R₁₁, R₁₂, R₁₃, R₁₄, Q and n are as defined above.

If not otherwise defined herein, R₁₂ means preferably (C₁₋₄)alkyl, such as methyl. R₁₄ means preferably alkyl such as methyl. If not otherwise defined herein, R₁₃ denotes preferably hydrogen, alkyl or acetyl, such as methyl.

In a preferred embodiment a compound of formula I is used wherein Y denotes sulfonyl and R₁, R₂, R₄, R₅, R₆, R₇, R₈, R₉, R₁₀, R₁₁, R₁₂, R₁₃, R₁₄, Q and n are as defined above.

In a particularly preferred embodiment a compound of formula I is used wherein Y denotes sulfonyl, R1 denotes amidino, R2 denotes a group of formula IId as defined above and R₄ denotes a group of formula IIIa as defined above, e.g. a compound of formula

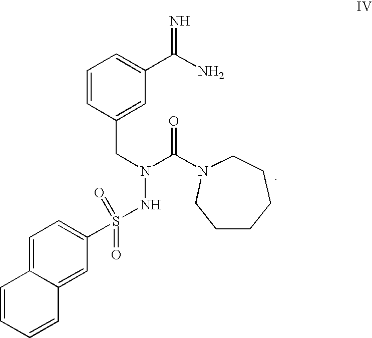

In an alternative particularly preferred embodiment a compound of formula I is used wherein Y denotes sulfonyl, R₁ denotes N-hydroxyamidino, R₂ denotes a group of formula IId as defined above and R₄ denotes a group of formula IIIa as defined above, e.g. a compound of formula

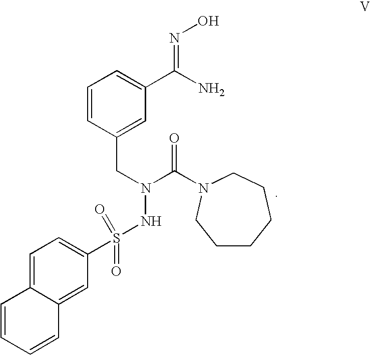

In a third particularly preferred embodiment a compound of formula I is used wherein Y denotes sulfonyl, R₁ denotes amidino, R₂ denotes a group of formula IId as defined above and R₄ denotes a group of formula IIId as defined above wherein n is 1 and R₁₄ denotes fluoro in ortho-position, e.g. a compound of formula

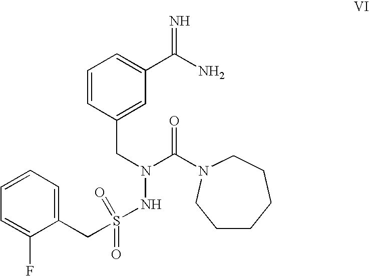

A compound of formula I, e.g. a compound of formula IV, V or VI, includes a compound of formulae I in any form, be it in free acid or free base form, in salt form, e.g. with a cation or with an acid, and/or a compound of formula I in the form of solvates, e.g. hydrates. A compound of formula I in salt form is for instance an acid addition salt, e.g. with an organic or organic acid, such as hydrochloric acid, hydrobromic acid or methylsulfonic acid. As well, a compound of formula I includes any tautomer or diastereo-isomer thereof, where they exist, for instance the syn- and anti-isomer with regard to a hyroxy-imino group.

Compounds of formula I and pharmaceutically acceptable salts thereof are partly known, e.g. from WO02/051824. It was also described therein that compounds of formula I may be useful as thrombin inhibitors.

Compounds of formula I can be prepared in analogy, e.g. according to known processes, for instance according to a process as described in WO02/051824.

It will be appreciated that reference to treatment herein is intended to include alleviation of established symptoms. Similarly, prevention includes any kind of prophylaxis, e.g. lowering the risk that certain symptoms will occur.

In a further aspect, the present invention is directed on a method of treatment or prevention, e.g. the treatment, of a cardiac arrythmia in a mammalian comprising administering a therapeutically effective amount of a compound of formula I as defined above to an individual in need thereof.

A mammalian as used herein includes a human being, particularly a human being suffering from a coronary heart disease, e.g. having in the medical history previous events of ischemic heart conditions such as myocardial infarction, angina pectoris or congestive heart failure, or a human being having in the medical history thrombotic events such as venous thrombosis, a thrombotic cerebral ischemia or a thrombotic apoplectic insult.

It has now surprisingly been found that compounds of formula I may be useful in the treatment or prevention of cardiac arrhythmias. The term “cardiac arrhythmia” refers to any disturbance in the rate, regularity, site of origin, or conduction of the cardiac electrical impulse, e.g. any deviation from a regular sinus rhythm. A cardiac arrhythmia can be an acute arrhythmia, a chronic arrhythmia or an intermittent, i.e. a paroxysmal arrhythmia and includes all kinds of non-regular heart rhythm, in particular ventricular arrhythmias as well as atrial, i.e. supraventricular arrhythmias. A cardiac arrhythmia includes for instance, ventricular or supraventricular fibrillation, ventricular or atrial fluttering, ventricular or supraventricular tachycardias, ventricular or supraventricular extrasystoly as well as asystoly. Thus, a compound of formula I may be useful in the treatment or prevention, e.g. the treatment, of ventricular fibrillation, supraventricular fibrillation, ventricular tachycardia, ventricular extrasystolia, supraventricular extrasystolia or asystolia. A compound of formula I may be particularly useful in the treatment or prevention, e.g. the treatment, of supraventricular or ventricular fibrillation, e.g. supraventricular fibrillation.

Cardiac Arrhythmias can be diagnosed as known in the art, e.g. by electrocardiogram (ECG).

Since compounds of formula I may also have anti-thrombotic properties the present invention concerns in a further aspect the use of a compound of formula I as defined above in the preparation of a medicament for the simultaneous treatment or prevention of cardiac arrhythmias and thrombosis. In a further aspect, the present invention is directed on a method for the simultaneous treatment or prevention, e.g. the treatment, of cardiac arrhythmia and thrombosis in a mammalian comprising administering a compound of formula I as defined above to an individual in need thereof. If a compound of formula I is used it may for instance not be necessary to administer simultaneously another antiarrythmic agent to treat or prevent cardiac arrhythmia while at the same time treating or preventing thrombosis. Cardiac arrhythmias may include those mentioned above whereas thrombosis includes any disease associated with the formation, development or presence of a thrombus and which may result in embolism and/or ischemia. The term “thrombosis” may thus include conditions associated with thrombus forming, in particular infarction of a blood vessel, be it arterial and/or venous blood vessels, by blood-clotting. Thrombosis includes for example thrombosis of peripheral vessels, infarction of coronary vessels, infarction of lung vessels or infarction of cerebral vessels such as in case of an ischemic apoplectic insult. A preferred use of the compounds according to formula I is therefore the preparation of a medicament for the simultaneous treatment or prevention of a supraventricular arrhythmia and the infarction of cerebral vessels, e.g. in connection with a cerebral ischemia for instance a thrombotic or ischemic apoplectic insult or a transient ischemic attack (TIA). A supraventricular arrhythmia is preferably a supraventricular fibrillation or atrial fluttering, e.g. a paroxysmal, persistent or a permanent form of supraventricular fibrillation or atrial fluttering.

In a particularly preferred embodiment a compound of formula I may be used in the preparation of a medicament for the treatment or prevention, e.g. the treatment, of arrhythmia absoluta, e.g. treatment of arrhythmia absoluta while simultaneously preventing or treating cerebral ischemia, in particular preventing ischemic apoplectic insults and/or transient ischemic attacks.

The antiarrhythmic properties of compounds of formula I were especially surprising because other antithrombotic compounds such as for instance argatroban or nadroparin are not known to have also antiarrhythmic properties. Since patients suffering from cardiac arrythmia, particularly from supraventricular arrhythmia such as atrial fibrillation have an increased risk for thrombotic diseases, e.g. apoplectic insult, it is highly desirable to treat them with both, an antiarrythmic and an antithrombotic agent. The use of compounds of formula I as defined above may offer the advantage to simultaneously treat or prevent both clinical factors, arrhythmia and thrombotic events, with a single active ingredient.

Further on, it has been observed that a compound of formula I as defined above may have a protective effect on cells, e.g. myocardial cells, against ischemia. During ischemia the cells are not sufficiently supplied with oxygen and therefore die after a while if the oxygen supply is not restored. The time during which an ischemia is tolerated by the cells depends on various conditions influencing supply and consumption rate of oxygen, e.g. temperature, type of tissue, oxygen supply from other vessels, metabolic activities, presence of hormones and other drugs etc. A compound of formula I as defined above may significantly prolong the tolerance time of cells, e.g. myocardial cells to ischemia. Thus, the critical time of an ischemia after which cells, e.g. myocardial cells will be able to revive may be extended by the use of a compound of formula I.

Therefore, the present invention is related in another aspect to the use of a compound of formula I as defined above in the preparation of a medicament for the treatment or prevention of diseases in association with ischemic heart conditions. In a further aspect, the present invention is directed on a method for the treatment of diseases associated with ischemic heart conditions in a mammalian comprising administering a compound of formula I as defined above to an individual in need thereof.

Such diseases in association with ischemic heart conditions are all diseases where the oxygen supply to the myocardial cells is or was partly or totally impaired including acute and chronic ischemic heart diseases, for example coronary heart disease, myocardial infarction, angina pectoris, congestive heart failure, acute cardiac arrest or states after successful resuscitation. Those conditions may be diagnosed by known methods, e.g. by ECG.

Compounds of formula I and pharmaceutically acceptable salts thereof may be administered to an individual in need of it in the form of a pharmaceutical composition. Suitable ways of administration include parenteral and enteral formulations as known in the art. Examples of suitable formulations of a compound of formula I are intravenous, oral, dermal, rectal, sublingual, endobronchial formulations, e.g. injection solutions, capsules, tablets, dermal patches, drinking solutions and sprays.

In a further aspect the present invention concerns a pharmaceutical composition for use in the treatment or prevention of cardiac arrhythmia comprising a compound of formula I as defined above and one or more pharmaceutically acceptable auxiliaries. The present invention concerns also a pharmaceutical composition for use in the simultaneous treatment or prevention, e.g. the treatment, of cardiac arrhythmia and thrombosis comprising a compound of formula I as defined above and one or more pharmaceutically acceptable auxiliaries. Another aspect of the present invention is a pharmaceutical composition for use in the treatment or prevention of diseases in association with ischemic heart conditions comprising a compound of formula I as defined above and one or more pharmaceutically acceptable auxiliaries. As well, the present invention concerns a method for treating or preventing, e.g. treating, cardiac arrhythmia, and in particular a method of treating arrythmia and thrombosis, wherein a pharmaceutical composition comprising a compound of formula I as defined above is administered to an individual in need of it.

Suitable pharmaceutically acceptable auxiliaries depend on the type of formulation and are those known in the art, such as solvents, binders, fillers, glidants. A suitable pharmaceutical composition may further comprise beside a compound of formula I one or more additional active ingredients. A preferred pharmaceutical composition is a parenteral solution or in the form of an oral dosage form, such as a tablet or a capsule.

A compound of formula I may be used alone or in combinations together with one or more other active ingredients. Combinations may be fixed dose combinations or the different active ingredients may be administered separately together or sequentially. Suitable active ingredients for combinations may include anticoagulants, e.g. vitamin K antagonists such as warfarin, fibrinolytics, antiplatelet agents and other antithrombotic agents as for instance cyclo-oxygenase inhibitors such as acetyl salicylic acid.

A compound of formula I may also be used in combination with pharmacologic or electric cardioversion, for instance during sufficient time, e.g. 3 to 4 weeks such as around 72 hours, before and/or after a cardioversion, in order to treat or prevent thrombotic events. Thus, compounds of formula I as defined above are useful for improving sinus rhythm in patients at risk for a recurrence of atrial fibrillation after cardioversion.

A compound of formula I may be administered to an individual in need of it once or several times within a certain period of time. Also a continuous application over a certain period of time is possible. The exact dosage and frequency of administration depends on the particular compound of formula I used, the particular condition being treated, the severity of the condition being treated, the route of administration, the age, weight, general physical condition of the particular individual, other medication the individual may be taking as is well known to those skilled in the art. A therapeutically effective dosage may be from about 0.01 mg to about 100 mg, e.g. from about 0.1 mg to about 10 mg, of compound of formula I per kg body weight and per day. In addition, the exact dosage and frequency of administration can be more accurately determined by measuring the blood level or concentration of the compounds of formula I in the patient's blood and/or the patients' response to the particular condition being treated. A therapeutically effective blood concentration may be for example from about 0.1 μMol/l to about 30 μM/l.

In further aspects the present invention concerns a method of treatment or prevention of a cardiac arrythmia in a mammalian comprising administering a therapeutically effective amount of a compound of formula I as defined above to an individual in need thereof.

In the same another aspect of the present invention is a method of simultaneous treatment or prevention of a cardiac arrythmia and thrombosis in a mammalian comprising administering a therapeutically effective amount of a compound of formula I as defined above to an individual in need thereof.

The following Examples illustrate but are not intended to limit the scope of the present invention.

### EXAMPLE 1

### Antiarrhythmic Effect and Increased Tolerance to Hypoxia Shown by Certain Aza-Phenylalanine Compounds in Reperfusion-Induced Arrhythmia

Wistar rats (230-330 g) are anaesthetized by intraperitoneal injection of urethane (0.7 ml 20% w/v solution/100 g body weight), followed by intraperitoneal injection of heparin (2500 IU/rat). Having opened the thorax a cannula filled with cold Krebs-Henseleit (K-H) solution is inserted into the ascending aorta. After that the heart is excised, it is connected to Langendorff's apparatus and perfused with K-H solution (K-H solution, in mM: 118.6 NaCl; 4.7 KCl; 11.1 glucose; 25 NaHCO₃; 1.66 MgSO₄; 1.2 NaH₂PO₄, 2.52 CaC₂; pH=7.4) under constant pressure (60 cm H₂O). Prior to use, the K-H perfusion solution is passed through a cellulose acetate filter (pore size 5 μm) to remove any particulate impurities. The perfusion solution is kept at 38.5° C. and gassed with 95% O₂ and 5% CO₂. Two silver (AgCl) electrodes are set on the surface of the heart in the direction of electrical axis of the heart for recording electrocardiogram. Electrodes are placed on the heart in such a position that a sufficient amplitude of P wave and QRS complex is obtained. The following experimental groups are studied: 1) isolated hearts are perfused with oxygenated K-H solution for 30 min followed by 40 min of global ischemia and followed by 50 min of reperfusion with oxygenated K-H solution; 2) isolated hearts are perfused with oxygenated K-H solution for 20 min, followed by 10 min perfusion with experimental drug dissolved in K-H and then the hearts are exposed to 40 min of global ischemia followed by 50 min reperfusion with experimental drug dissolved in K-H. The used experimental drugs are compounds of formulae IV, V and VI as defined above. As a positive control direct thrombin inhibitor argatroban and low molecular weight heparin nadroparin are used. The hearts are monitored for the duration of proarrhythmic events according to the Lambeth convention (Walker et al., Cardiovascular Res 1988; 22: 447-455). Lactate dehydrogenase (LDH) release rate is determined spectophotometrically (Wroblevski & LaDue, Proc Exp Biol Med 1955; 90: 210-213) in the coronary effluent and is expressed in μkat per minute and per weight of the heart (Grasic Kuhar et al. Eur J Pharmacol 2004; 488: 137-46).

Table 1 shows the duration of ventricular tachycardias, ventricular fibrillations and the sum of all proarrhytmic events including ventricular tachycardia, ventricular fibrillation, extrasystolia and asystolia in each experimental group.

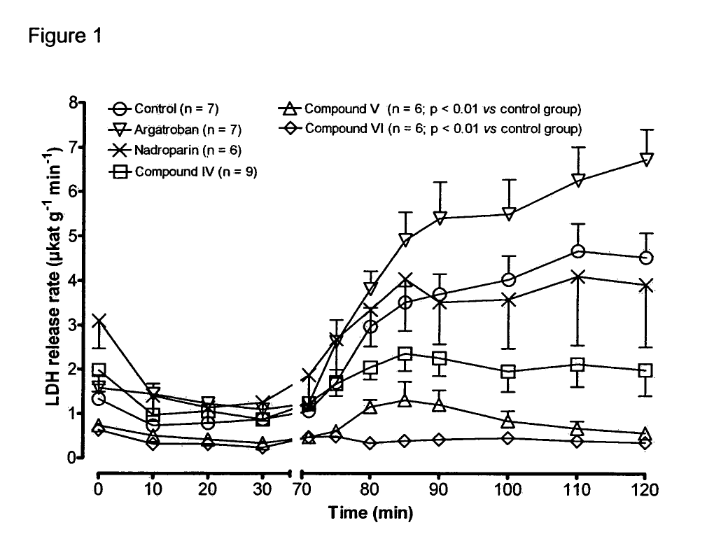

FIG. 1 shows LDH release rate in each experimental group.

TABLE 1
Duration of ventricular tachycardia (VT), ventricular fibrillation (VF) and all
proarrhythmic events including VT, VF, extrasystolia and asystolia in isolated rat
hearts subjected to 40-min ischemia.

| Treatment | | Duration VT | Duration VF | Duration of all |
|---|---|---|---|---|
| (concentration) | n | (s) | (s) | proarrhythmic events (s) |
| Control | 7 | 761.3 ± 137.9 | 858.0 ± 229.1 | 1996.0 ± 338.8 |
| Compound IV (1 μM) | 9 | 87.2 ± 53.3** | 78.6 ± 42.6* | 353.3 ± 148.1** |
| Compound V (1 μM) | 6 | 60.0 ± 30.8* | 149.3 ± 86.4 | 278.3 ± 104.2* |
| Compound VI (1 μM) | 6 | 99.5 ± 77.95* | 596.0 ± 469.4 | 810.2 ± 461.4 |
| Argatroban (1 μM) | 7 | 828.7 ± 233.7 | 583.6 ± 228.6 | 1731.0 ± 445.0 |
| Nadroparin (51.6 IU | 6 | 599.5 ± 373.6 | 500.3 ± 426.4 | 1194.0 ± 604.2 |
| anti Xa/l) | | | | |

In control group hearts are perfused with Krebs-Henseleit (K-H) solution only.
Results are presented as means standard error of mean.
For the statistical analysis data are log transformed and compared to control group using ANOVA with Dunnett's multiple comparison test,
*p < 0.05
**p < 0.01.
n: number of subjects.

FIG. 1: Lactate dehydrogenase (LDH) release rate (μkat g⁻¹ min⁻¹) in isolated rat hearts subjected to 40-min ischemia (between 30 and 70 minute). In control group the hearts are perfused with Krebs-Henseleit (K-H) solution only. In other experimental groups the hearts are perfused with K-H solution containing compound IV (c=1 μM), compound V (c=1 μM), compound VI (c=1 μM), nadroparin (51.6 IU anti Xa/I) and argatroban (c=1 μM). Results are presented as means±standard error of mean. Statistically significant difference between treatment groups is determined by calculating the area under the curve followed by ANOVA with Dunnett's multiple comparison test.

### EXAMPLE 2

### Pharmaceutical Composition

100 mg of compound of formula V is dissolved in 100 ml of sterile pyrogen-free water under mixing. Then, the solution is filled into 20 ampoules under sterile conditions to obtain ampoules each containing 5 ml of injection solution for the treatment of cardiac arrythmia. Each ampoule is packed into a box together with a leaflet mentioning cardiac arrythmia as an indication for the injection solution.

### EXAMPLE 3

### Antiarrhythmic Effect Shown by a Compound of Formula Iv on Reperfusion-Induced Arrhythmia

Hearts from male Wistar rats (300-370 g) anesthetized with diethyl-ether inhalation were perfused with Krebs-Henseleit buffer at 37° C. gassed with carbogen in Langendorff mode as described (Onody A et al., Cardiovasc Res. 2003, 58: 663-670). After an initial 15 min aerobic, normothermic perfusion period, hearts are subjected to 25-min of global, no-flow, normothermic ischemia, followed by 30 min normoxic, normothermic reperfusion. Measured parameters were heart rate and incidence of reperfusion-induced ventricular fibrillation (VF) and ventricular tachycardia (VT). Epicardial electrocardiogram was recorded throughout the experimental protocol to determine heart rate, VF and VT according to the Lambeth conventions (Walker et al., Cardiovasc Res. 1988; 22: 447-455) as described (Csonka et al., 2003; J Cardiovasc Pharmacol 41: 916-922; Ferdinandy et al., Cardiovasc Res. 1995; 58: 663-670.). Recording and evaluation of the ECG was performed using the SPEL Advanced Isosys system (Experimetria Ltd, Budapest, Hungary). Experimental groups were designed as follows: control, solvent (DMSO 500 μL/L), compound of formula IV at 1 μM (+DMSO 500 μL/L), compound of formula IV at 10 μM (+DMSO 500 μL/L), positive control: nifedipine at 0.5 μM (+DMSO 500 μL/L). Animals were randomly assigned to the different groups, however, perfusions were done in 4 parallel experiments within a group (n=12 in each group). The perfusion medium contained the drugs throughout the entire perfusion protocol in the treated groups. HR was compared to the solvent-treated group using ANOVA followed by Dunn's test. The incidence of VF and VT were compared with Chi² test in all groups, than each group was compared to the solvent-treated group using the Fischer's exact test.

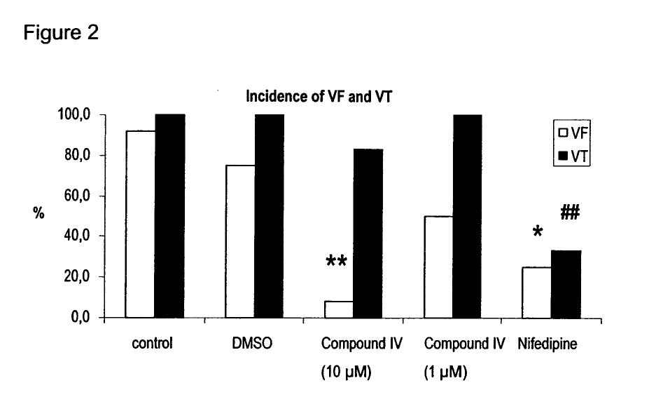

FIG. 2 shows the incidence of ventricular fibrillation (VF) and ventricular tachycardia (VT) in isolated rat hearts subjected to global ischemia. Compound of formula IV was tested at 10 μM (Compound of formula IV, 10 μM) and 1 μM (Compound of formula IV, 1 μM), nifedipine was tested at 0.5 μM. Chi² test followed by the Fischer's exact test. * p<0.05; ** p<0.01; ## p<0.01 (all groups compared to DMSO group)

It is shown that that the incidence of VF was concentration-dependently decreased by compound of formula IV, however the incidence of VT and the heart rate (HR) was not significantly changed. The positive control nifedipine significantly decreased the incidence of both VF and VT.

Thus, Example 3 proves that compound of formula IV has a strong antiarrhythmic effect upon reperfusion.

1-10. (canceled)

11. A method of treating or preventing cardiac arrhythmias in a subject, wherein an effective amount of a compound of formula 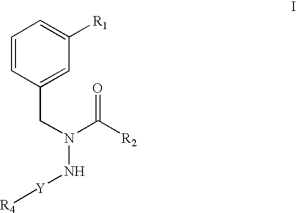
or a pharmaceutically acceptable salt or a solvate thereof is administered to the subject, whereby in formula I R₁ denotes amino, alkylamino, dialkylamino, amidino, alkylamidino, N-hydroxyamidino, or N-alkoxyamidino, R₂ denotes a group of formula 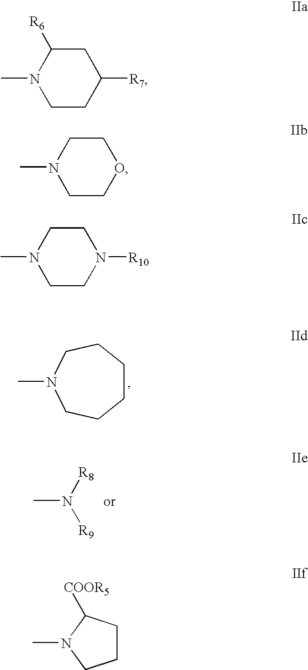
wherein R₅ denotes hydrogen, alkyl or aryl,
R₆ and R₇ each independently denote hydrogen, alkyl or —COOR₁₁,
R₈ and R₉ each independently denote alkyl or cycloalkyl,
R₁₀ denotes hydrogen, alkoxycarbonyl or alkylsulfonyl, and
R₁₁ denotes hydrogen or alkyl,
Y denotes carbonyl or sulfonyl
R₄ denotes alkyl, alkoxy or a group of formula 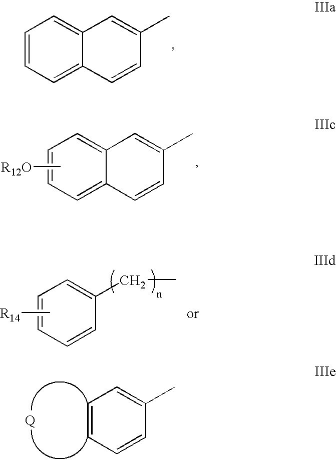
wherein R₁₂ denotes alkyl, R₁₄ denotes alkyl or halogen, n represents an integer number from 0 to 3, and Q forms together with the phenyl ring to which it is attached a 5- to 7-membered alicyclic ring having at one position a nitrogen atom substituted by R₁₃, wherein R₁₃ denotes hydrogen, alkyl or acyl.

12. The method according to claim 11, wherein the cardiac arrhythmia is selected from the group consisting of ventricular fibrillation, ventricular fluttering, supraventricular fibrillation, atrial fluttering, ventricular tachycardia, ventricular extrasystolia, supraventricular extrasystolia and asystolia.

13. The method according to claim 12, wherein the arrhythmia is a supraventricular fibrillation or atrial fluttering.

14. A method of simultaneously treating or preventing cardiac arrhythmias and thrombosis in a subject, wherein an effective amount of a compound of formula I as defined in claim 11 is administered to the subject.

15. A method of treating or preventing diseases in association with ischemic heart conditions in a subject, wherein an effective amount of a compound of formula I as defined in claim 11 is administered to the subject.

16. The method according to claim 11, wherein the compound of formula I is selected from the group consisting of compounds of formula

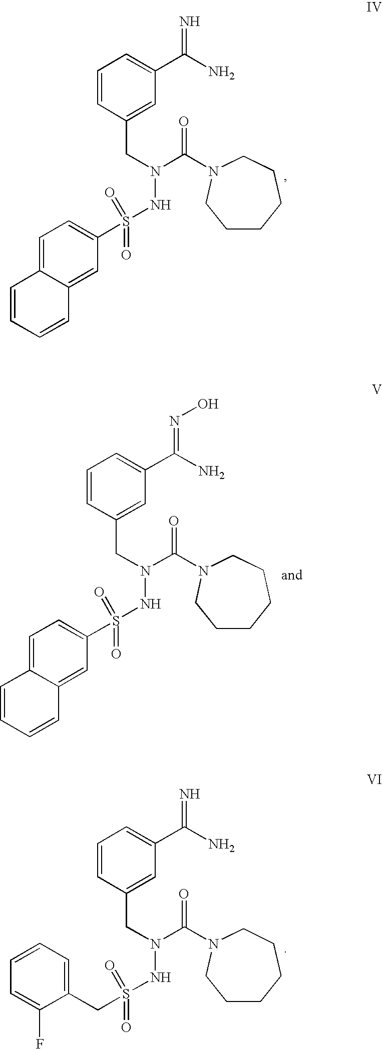

17. The method according to claim 14, wherein the compound of formula I is selected from the group consisting of compounds of formula

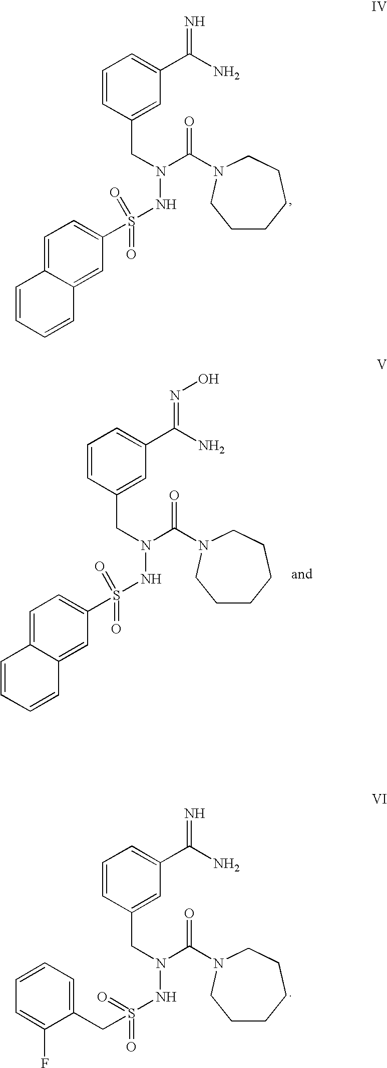

18. The method according to claim 15, wherein the compound of formula I is selected from the group consisting of compounds of formula

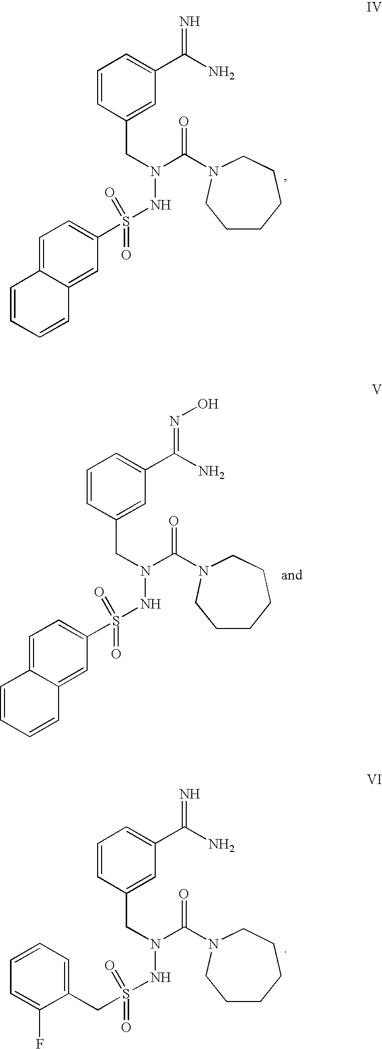

19. A pharmaceutical composition for use in the treatment or prevention of cardiac arrhythmia comprising a compound of formula I 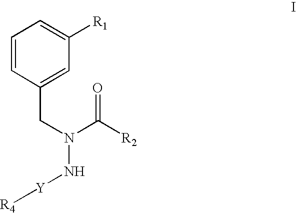
or a pharmaceutically acceptable salt or a solvate thereof, whereby in formula I R₁ denotes amino, alkylamino, dialkylamino, amidino, alkylamidino, N-hydroxyamidino, or N-alkoxyamidino, R₂ denotes a group of formula 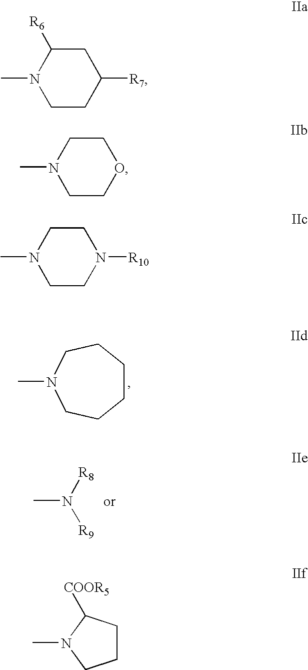
wherein R₅ denotes hydrogen, alkyl or aryl,
R₆ and R₇ each independently denote hydrogen, alkyl or —COOR₁₁,
R₈ and R₉ each independently denote alkyl or cycloalkyl,
R₁₀ denotes hydrogen, alkoxycarbonyl or alkylsulfonyl, and
R₁₁ denotes hydrogen or alkyl,
Y denotes carbonyl or sulfonyl
R₄ denotes alkyl, alkoxy or a group of formula 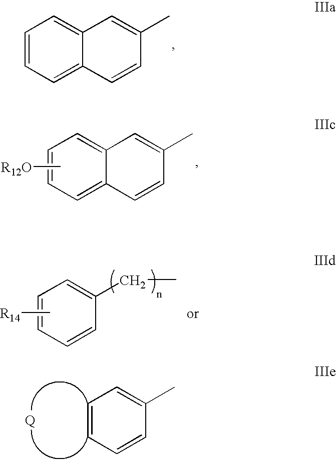
wherein R₁₂ denotes alkyl, R₁₄ denotes alkyl or halogen, n represents an integer number from 0 to 3, and Q forms together with the phenyl ring to which it is attached a 5- to 7-membered alicyclic ring having at one position a nitrogen atom substituted by R₁₃, wherein R₁₃ denotes hydrogen, alkyl or acyl and one or more pharmaceutically acceptable auxiliaries.

20. A pharmaceutical composition for use in the simultaneous treatment or prevention of cardiac arrhythmia and thrombosis comprising a compound of formula I: 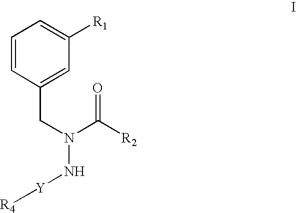
or a pharmaceutically acceptable salt or a solvate thereof, whereby in formula I R₁ denotes amino, alkylamino, dialkylamino, amidino, alkylamidino, N-hydroxyamidino, or N-alkoxyamidino, R₂ denotes a group of formula 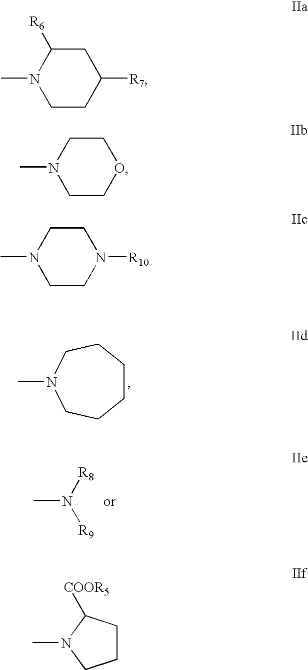
wherein R₅ denotes hydrogen, alkyl or aryl,
R₆ and R₇ each independently denote hydrogen, alkyl or —COOR₁₁,
R₈ and R₉ each independently denote alkyl or cycloalkyl,
R₁₀ denotes hydrogen, alkoxycarbonyl or alkylsulfonyl, and
R₁₁ denotes hydrogen or alkyl,
Y denotes carbonyl or sulfonyl
R₄ denotes alkyl, alkoxy or a group of formula 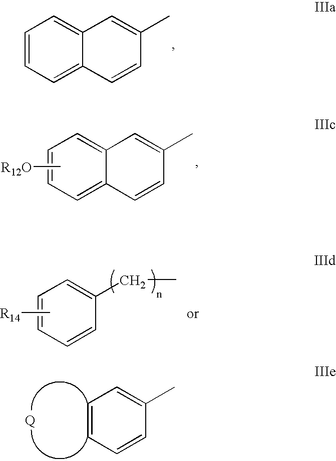
wherein R₁₂ denotes alkyl, R₁₄ denotes alkyl or halogen, n represents an integer number from 0 to 3, and Q forms together with the phenyl ring to which it is attached a 5- to 7-membered alicyclic ring having at one position a nitrogen atom substituted by R₁₃, wherein R₁₃ denotes hydrogen, alkyl or acyl and one or more pharmaceutically acceptable auxiliaries.

21. The pharmaceutical composition according to claim 19, wherein said pharmaceutical composition is in the form of a parenteral solution or in the form of an oral dosage form.

22. The pharmaceutical composition according to claim 20, wherein said pharmaceutical composition is in the form of a parenteral solution or in the form of an oral dosage form.
